(19) United States
(12) Patent Application Publication
(10) Pub. No.: US 2018/0110533 A1
(43) Pub. Date: Apr. 26, 2018

(54) INFUSION, DILATION AND ASPIRATION CATHETER (IDAC)

(71) Applicant: Gerhard-Friedrich HORAK, Vienna (AT)

(72) Inventor: Gerhard-Friedrich HORAK, Vienna (AT)

(21) Appl. No.: 15/559,356

(22) PCT Filed: Mar. 15, 2016
(86) PCT No.: PCT/EP2016/055602

§ 371 Date: Sep. 18, 2017

(30) Foreign Application Priority Data
Mar. 17, 2015 (DE) 202015002060.7

(51) Int. Cl. A61B 17/22; A61M 25/00

(52) U.S. Cl. CPC A61B 17/22; A61M 25/0068; A61B 2017/22074; A61B 2017/22079; A61M 2025/1004; A61B 2217/005; A61B 2217/007; A61M 2025/0004; A61B 2017/22084

(57) ABSTRACT

A system (1) comprises an aspiration catheter (2), which is guided by way of a guidewire (3) to a thrombotic point in a blood vessel. A collapsed infusion balloon (4) is located inside the aspiration catheter (2), which has an interior/inner volume (40) delimited by a wall (41), and a delivery catheter (42). The delivery catheter 42 is intended for delivering saline solution (salt solution) for rinsing or for delivering thrombolytic medication into the interior 40. The balloon comprises a plurality of openings which have a diameter between 10 μm and 100 μm in order to enable an atraumatic administration of fluid.

### PRIOR ART

It is known that thrombi or thrombotic material can be removed from blood vessels by means of so-called aspiration or suction catheters. To this end, the aspiration catheter is introduced and guided to the thrombotic site. Thrombotic material can then be removed from the blood vessel via the aspiration catheter. The aspiration catheter is advanced for this purpose until its distal end reaches the region from which thrombotic material is to be aspirated. The material is now aspirated through the lumen of the suction catheter, e.g. by means of a Luer Lock syringe.

A disadvantage of this method and the catheters designed for this purpose is that the thrombotic material agglomerated in the blood vessel can only be detached from the vessel wall with difficulty in some circumstances. Moreover, the detached agglomerates can be of relatively large volume, so that targeted aspiration through the aspiration catheter can be difficult. In addition, there is the risk that detached thrombotic material will be removed from the site of the procedure and deposited elsewhere or will be discharged into the vascular system and possibly occlude another one. There is therefore a high risk of further thrombotic occlusions (e.g. of the cerebral artery).

On the other hand, infusion catheters are known, by means of which are administered saline solutions for rinsing or medications (e.g. thrombolytic medications) for removing dissolved thrombotic material or dissolving thrombotic material (debris/plaque) from the vessel wall.

A disadvantage of these techniques is that, on one hand, the effect of the medication which is administered via a conventional infusion catheter, e.g. microcatheter consisting essentially of a lumen with a distal opening, is low due to dilution effects. For this reason, a large amount of the medication must be delivered on the other hand. However, in these circumstances, medication which has been administered in an uncontrolled manner or detached thrombi can enter other regions of the circulatory system, a fact which can lead to significant side effects such as stroke.

### OBJECT OF THE INVENTION

Proceeding therefrom, an object of the invention is to provide a catheter which solves the aforementioned problems and ensures a rapid and uncomplicated removal of thrombotic material.

### Technical Solution

This object is achieved by a system according to one of claim 1 or 13 and by an infusion balloon according to claim 10. Further preferred exemplary embodiments result from the characteristics of the dependent claims.

The system for removing thrombotic material from a blood vessel comprises an aspiration catheter and an infusion member, wherein the infusion member has a delivery catheter for delivering a fluid and an expandable balloon connected to the delivery catheter, wherein the balloon has openings and/or at least one (semi)permeable wall region through which the fluid can pass, wherein the infusion member can be movably accommodated in and moved through a first lumen of the aspiration catheter.

The problem with conventional systems is that, when a thrombolytic medication is delivered into a thrombotic vessel, incompletely dissolved thrombotic material and/or debris/plaque can be transported further and in certain circumstances may trigger, e.g. a stroke in the brain, haemorrhagic insult, or other complications, in a different place.

The inventive solution makes it possible for the balloon, which is initially disposed within the first lumen of the aspiration catheter, to be introduced along with the aspiration catheter into the blood vessel. Subsequently, the balloon is pushed out of the first lumen (e.g. by way of a guidewire for the infusion member) to where it is level with the site in the blood vessel which is occupied by thrombi and constricted. Thus, on one hand, thrombi at the vessel wall become immobilised and, on the other, are dissolved by the administered fluid, but are unable to migrate uncontrollably.

The balloon is then dilated via the delivery catheter by increasing the pressure and the amount of liquid, e.g. liquid medication (thrombolytic medication).

Once a certain pressure has been attained, the balloon has reached a size at which the outer side of its wall bears against the wall of the blood vessel and the thrombi are immobilised at the site.

Once the medication has reached a certain internal pressure, the wall and/or the openings of the balloon become permeable to it.

When the outer skin/outer side of the wall of the balloon bears against the wall of the blood vessel, the external diameter of the balloon (relative to a projection of the balloon perpendicular to the vessel wall) is greater than the internal diameter of the first lumen of the aspiration catheter. The balloon is then exerting a substantial pressure on the vessel wall. Thus, on one hand, dissolved thrombi are at least partly immobilised by the balloon and cannot be transported away; on the other, the close contact and variable contact pressure of 2 to 10 bar, especially of at least 4 bar, especially of at least 6 bar between the outer wall of the balloon and the vessel wall, leads to extremely effective administration of the medication.

The openings and/or the (semi-)permeable wall region are especially configured such that atraumatic administration of the fluid takes place after a predetermined fluid pressure has been reached.

For its part, the target contact pressure is achieved by a high internal pressure of the liquid within the balloon of 2 to 10 bar, especially of at least 4 bar, especially of at least 6 bar. In order that sufficiently high internal pressures may be achieved despite the openings in the balloon, the openings must be relatively small, especially between 10 μm and 100 μm, preferably between 10 μm and 60 μm, especially preferably between 20 μm and 50 μm.

On the other hand, effective administration of the medication is achieved by way of a high number of small openings so that a kind of liquid film and/or a slow wetting of the outer side of the balloon occurs and an internal pressure between 2 and 10 bar is maintained. The balloon has an area density of openings (i.e. openings per unit area) of the said size of at least 25 openings per cm², especially at least 50 openings per cm², especially at least 100 openings per cm², especially at least 500 openings per cm², especially at least 1000 openings per cm², averaged over the entire outer surface of the balloon (in the dilated state) in each case. Especially, openings are provided both in the section close to the proximal end and in the section adjacent to the distal end. There are substances which in high concentration will dissolve old and/or large thrombi. When systemically administered in high concentrations, however, these medications could lead to bleeding and other complications.

The balloon can have at least 50 openings, especially at least 100 openings, especially at least 500 openings, especially at least 1000 openings.

The balloon can vary in size in terms of its length, diameter, volume and/or surface area—in the dilated state in each case. Especially, the length l can be between 10 mm and 20 mm for balloons for the coronary region, between 10 mm and 300 mm for balloons for the peripheral region. The diameter can be between 1 mm and 4 mm for balloons for the coronary region, and between 1 mm and 8 mm for the peripheral region. In the non-dilated state, the diameter of the balloon can be, e.g. about 1 mm.

The infusion member is especially disposed so as to be axially displaceable in the first lumen of the aspiration catheter. The infusion member itself can comprise a guide member, e.g. a guidewire, for pushing the infusion member within and out of the first lumen.

The balloon is configured as a microballoon, which—in the non-dilated state—can be accommodated in the first lumen of the aspiration catheter and passed through the first lumen.

The balloon is only permeable to the medication above a certain internal pressure, e.g. 2 bar. With the aid of a pressure syringe, higher internal pressures of up to 10 bar can also be achieved. That is to say, medication is administered only after a certain pressure/volume has been reached, especially only when the outer skin is already bearing against the wall of the blood vessel.

The aspiration catheter has especially a proximal end and a distal end as well as a longitudinal axis, and the distal end is preferably bevelled.

The system can include a guide member immobilised on the aspiration catheter.

The aspiration catheter preferably has a second lumen in which the guide member (e.g. guidewire) is embedded at least sectionwise.

The object is achieved by a balloon for a system as described, wherein the balloon has a plurality of openings which have a diameter between 10 μm and 100 μm, especially between 10 μm and 60 μm, especially between 20 μm and 50 μm.

The balloon can have a plurality of openings with an average area density (i.e. openings per unit area) of at least 25 openings per cm², especially at least 50 openings per cm², especially at least 100 openings per cm², especially at least 500 openings per cm², especially at least 1000 openings per cm².

The balloon can have at least 50 openings, especially at least 100 openings, especially at least 1500 openings, especially at least 1000 openings. The balloon can also be constructed as a semipermeable balloon.

Preferably, the arrangement of the openings can also be determined by their average spacing. The spacing between adjacent openings 43 averages less than 2 mm, preferably less than 1.4 mm, especially less than 0.7 mm, especially preferably less than 0.45 mm, especially less than 0.3 mm. These values result from the density of the openings per unit area, as described above. The scope of the application, however, also seeks protection for arrangements of openings in which the area density as stated above is realised only over a certain region of the surface of the balloon. However, in these surface regions, the surface density of the openings (locally) should be as indicated above. This means that the average spacing between adjacent openings, insofar as the openings have a nearest neighbour, lies in the above-mentioned ranges in these surface regions.

With the aid of the absolute number of openings, the proportion of the surface of the balloon which must have openings for the purposes of the invention can be calculated.

As a result of the design of the balloon and its openings, effective administration of the active solution is achieved. The medication acts directly at the site to be treated. After delivery to the thrombus, the medication spreads throughout the circulatory system, where it acts systemically.

Especially, the balloon is made of nylon, polyethylene terephthalate (PET), polyamide, polyethylene, and/or polyether block amides (PEBA). It can have openings as described above, or at least be partly made of (semi-)permeable material which allows diffusion of the liquid and/or penetration of the membrane at a corresponding internal pressure.

The openings are preferably produced by laser treatment (e.g. laser cutting, production of punctiform openings). However, on an individual basis, the openings can also be formed, e.g. mechanically.

Following dissolution of the thrombi by targeted, selective infusion of medication and dilation, the internal pressure and thus the volume of the balloon are reduced to such an extent that the latter can be withdrawn through the first lumen of the aspiration catheter. To this end, e.g., a negative pressure is applied which causes the balloon to collapse or fold together.

When the balloon is retracted through the first lumen, it essentially terminates with the first lumen of the suction catheter. This creates a vacuum (negative pressure) or a suction effect (Windkessel effect), which draws further, already dissolved thrombotic material into the aspiration catheter in this early phase and thus removes it from the blood vessel.

Once the balloon has been completely retracted from the first lumen, an apparatus for generating a vacuum or a suction action is attached to the proximal end of the aspiration catheter, e.g. an aspiration syringe (e.g. a Luer Lock syringe) to remove as much of the remaining dissolved thrombotic material as possible.

Moreover, the permeability is reversible or exhibits hysteresis. In a reversible configuration, the openings close again if the first pressure drops below a specific value, with the result that the volume can be further reduced to a desired value.

In the case of hysteresis, the permeability disappears upon reaching a second predetermined pressure which does not have to correspond to the first predetermined pressure.

However, it can also be that the permeability of the balloon does not change or changes only quantitatively when the pressure is reduced. By adapting/reducing the fluid pressure in the interior to a third predetermined value, a dynamic equilibrium can be produced via corresponding medication delivery into the balloon, i.e. the balloon loses exactly as much medication as is delivered, thus retaining its volume.

The method comprises the following steps:

Within the scope of the invention, a method is claimed which has been implemented especially in connection with the inventive system as described in this application.
a) Provision of a system as described above;
b) Introduction of the system into a blood vessel and manoeuvring of the distal end of the aspiration catheter to the site to be treated (the infusion member being disposed within the first lumen of the aspiration catheter);
c) Moving of the balloon out of the first lumen of the aspiration catheter to the level of the site to be treated;
d) Dilation of the balloon by introducing a liquid and/or increasing the liquid pressure in the interior of the balloon, whereby, once a certain internal pressure is reached, the liquid passes through the openings formed in the balloon wall and outwards through the balloon (i.e. atraumatic administration of the liquid to the site to be treated);
e) Deflation and retraction of the balloon through the first lumen; and
f) Aspiration, i.e., suction of thrombotic material out of the blood vessel through the first lumen of the aspiration catheter.
   In step d), the balloon is preferably expanded until it bears or presses against the inner wall of the blood vessel at a predetermined pressure.

Further method steps have already been described and can be combined with process steps a) to f), in order to describe particular embodiments of the method.

A further inventive system for the removal of thrombotic material from a blood vessel comprises an aspiration catheter which has a first lumen for aspirating thrombotic material from a blood vessel and a guide member for introducing the aspiration catheter into the blood vessel, wherein the guide member (configured as an aspiration catheter and/or microcatheter) has a proximal end and a distal end, as well as a third lumen through which liquids can be transported from the proximal end of the guide member to the distal end of the guide member.

The guide member preferably has at least one infusion opening, which is formed in the region of the distal end of the guide member.

The aspiration catheter has especially a longitudinal axis and the distal end may be bevelled.

The guide member is immobilised especially on the aspiration catheter.

The distal end of the guide member can protrude over the edge of the opening of the second lumen of the aspiration catheter.

The cross-section of the first lumen is especially larger than the cross-section of the second lumen.

The guide member can be configured as a microcatheter and thus act as an infusion member.

### BRIEF DESCRIPTION OF THE DRAWINGS

Further characteristics and advantages of the present invention will become apparent from the following description of preferred embodiments using the figures. These show in

FIG. 1 A representation of an embodiment of the inventive system in a first phase of the inventive method;

FIG. 2 A representation of the embodiment of FIG. 1 in a second phase of the inventive method;

FIG. 3 A representation of the embodiment of FIG. 1 in a third phase of the inventive method;

FIG. 4 A representation of the embodiment of FIG. 1 in a fourth phase of the inventive method;

FIG. 5 A representation of the embodiment of FIG. 1 in a fifth phase of the inventive method;

FIG. 6 A representation of an embodiment of the inventive infusion balloon;

FIG. 7 A representation of a detail of the inventive infusion balloon from FIG. 6;

FIG. 8 A second embodiment of the inventive infusion, dilation and aspiration catheter;

FIG. 9 A cross-section A-A of the aspiration catheter as per FIG. 8.

### DESCRIPTION OF PREFERRED EMBODIMENTS

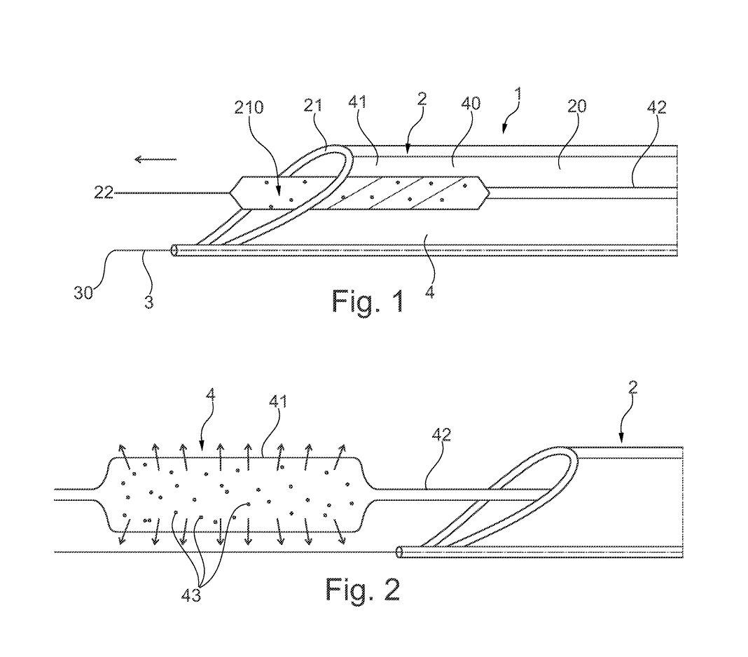

FIG. 1 shows a system 1 as per the present invention during a surgical procedure in a blood vessel B. The blood vessel B can especially be a coronary vessel, on the wall of which a thrombus B (plaque) has deposited and forms a constriction to be treated.

The system 1 has an aspiration catheter 2 with a first lumen 20, a distal end 21 (with a distal opening 210), and a proximal end with a proximal opening (not shown). A central axis 22 extends longitudinally between the proximal end and the distal end 21.

The system 1 also has a guidewire 3 which also has a proximal end (not shown) and a distal end 30. The guidewire 3 is disposed in a second lumen of the aspiration catheter 2. The guidewire 3 is immovably connected to the aspiration catheter 2, so that the guidewire 3 is not longitudinally displaceable with respect to the aspiration catheter 2. It protrudes beyond the distal opening of the second lumen. The area defined by the edge of the opening 210 of the aspiration catheter 2 is inclined at an angle of about 45 DEG with respect to the axis 22.

Inside the aspiration catheter 2 there is an infusion member, with an infusion balloon 4 which at this juncture is folded together and which has an interior chamber/interior volume 40 bounded by a wall 41 and a delivery catheter 42. The balloon 4 is folded such that it has a low profile, i.e. the cross-section is such that the balloon can be disposed in the aspiration lumen and can be moved through it.

The delivery catheter 42 is intended for delivering saline solution (salt solution) for rinsing or for delivering thrombolytic medication into the interior 40. Optionally (not shown), the balloon 4 has an actuating member, e.g. a separate guidewire, so that it can be moved back and forth relative to the aspiration catheter 2 in the direction of the longitudinal axis 22.

In the first phase of the procedure shown in FIG. 1, the catheter 2, together with the balloon 4 (which is still folded), which is disposed in the first lumen 20 of the aspiration catheter 2 (namely in the aspiration lumen), is introduced into the blood vessel by actuation of the guidewire 3 until it reaches its destination position. The destination position is reached when the opening 210 is disposed at a certain distance in front of the thrombus T. In this position, the distal end 21 of the catheter 2 reaches the constriction to be treated (caused by the thrombus).

In the second step shown in FIG. 2, the balloon 4 is pushed out of the first lumen 21 until it is approx. at the level of the thrombotic site to be treated. There, it is unfolded and/or dilated by introducing a medication via the delivery catheter 42. To this end, a pressure syringe is preferably used which can generate correspondingly high and defined internal pressures in the interior 40 of the balloon 4. When the internal pressure of the medication has reached a certain threshold value, the medication M is administered from the interior 40 of the balloon 4 into the blood vessel B via a plurality of micro-openings 43. Due to the small diameter of the openings of 10 μm to 100 μm, the medication M forces its way only slowly out of the balloon interior 40. The medication more or less wets the outer side of the balloon wall 41. At the same time, the configuration of the openings 43 with the said diameters ensures that a relatively high pressure of 2 bar to 10 bar is maintained in the interior 40. As a result, the balloon wall 41 presses with high pressure against the region of the wall of the blood vessel B, on which thrombotic material is deposited.

Due to the nature of the application—high contact pressure of the balloon 4 against the vessel wall combined with slow exit of the medication M from the plurality of small openings 43—atraumatic and at the same time extremely effective and intensive administration of the medication M is ensured at precisely the point T to be treated. The increase in the effect of the medication can be in the 4-digit range as compared with intravenous administration. The medication M unfolds its effect (practically only) at the site of application, e.g., by dissolving the thrombus T.

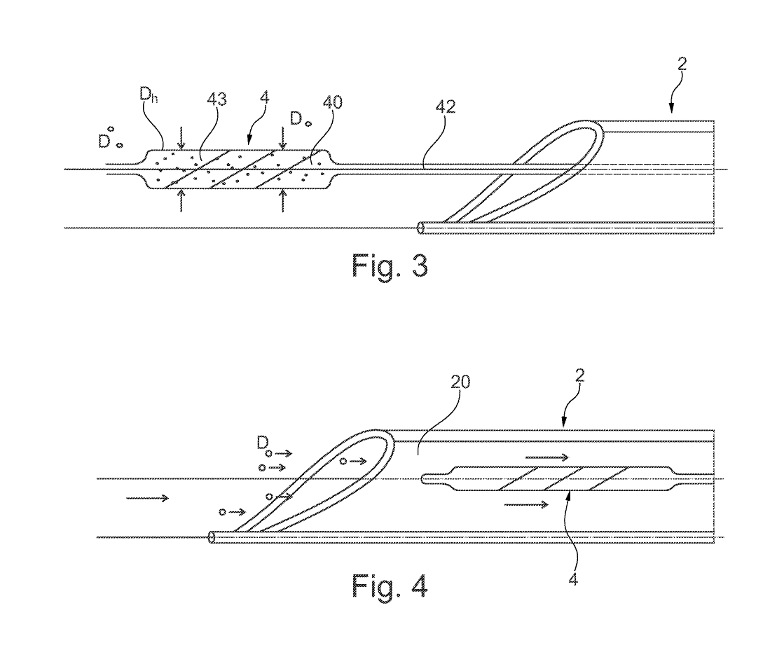

FIG. 3 shows the next phase of the use of system 1 in which the volume of the interior 40 is reduced by lowering the pressure of the medication M in the interior 40 of the balloon 4. The balloon body 40 collapses and more or less returns to its original dimensions. The openings 43 can, but need not, close reversibly (or along a hysteresis loop), such that the medication is no longer administered after a certain internal pressure is reached.

The administered medication M now unfolds its effect, a fact which is indicated by the dissolution and/or reduction in size of thrombotic material D. Any thrombotic material, IX resting against the outer side of the wall 41, can adhere to it. Additional thrombotic material D can be present in dissolved form in blood vessel B.

Subsequently, as shown in FIG. 4, the balloon 4 is drawn through the opening 210 and the first lumen 21 as far as the proximal end of the aspiration catheter 4.

The balloon is folded such that it has a low profile, i.e. the cross-section is such that the balloon can be fitted into and retracted through the aspiration lumen.

Through the act of retraction, the balloon 4 can produce a vacuum or so-called suction effect, indicated by the arrows of the debris particles D which follow the suction (Windkessel effect) and remove thrombotic material D, from the blood vessel B through the first lumen 20.

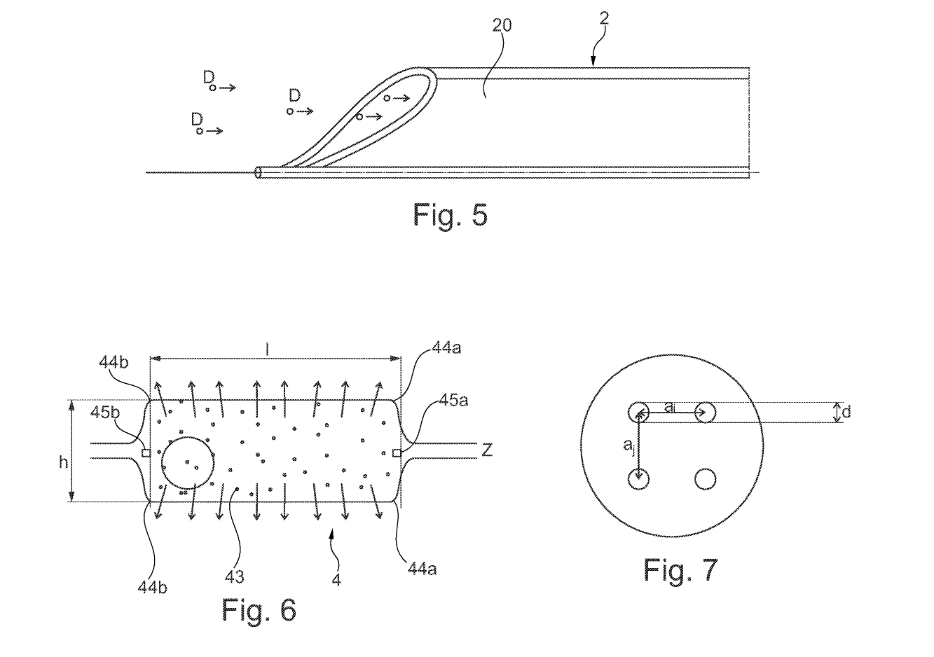

In the next step as per FIG. 5, the thrombotic material D is aspirated via the first lumen 20. For this purpose, a suction apparatus (not shown) is attached to the proximal end of the aspiration catheter 4. In this way, the thrombotic material D still remaining in the blood vessel B is largely removed.

Subsequently, administration is concluded and the system 1 can be removed from the blood vessel B.

FIG. 6 schematically shows the structure of an inventive microballoon/infusion balloon 4 in a dilated state. In the dilated state, the diameter h is between 1 mm and 4 mm. In addition, the balloon volume 40 has a length l of 10 mm to 20 mm. Especially, the plurality of openings 43 (indicated by the plurality of points, only one of which is marked with the reference numeral 43) is formed in the proximal as well as in the distal partial region of the balloon body 4. The liquid introduced into the balloon 4 is especially intended to act in the distal region of the balloon as well.

Upon delivery and increase of the internal pressure of the medication M or saline solution, the internal volume 40 of the balloon 4 increases until, during treatment, the outer side of the wall and/or skin 41 of the balloon 4 makes contact with the wall of the blood vessel, enclosing thrombotic material there and/or pressing it against the wall.

The balloon has the following geometry: In the inflated state, the balloon is essentially cylindrical or tubular with a central axis Z and a radius of h/2. At both ends, the balloon 4 has an annular shoulder 44a and 44b, respectively. The annular shoulder has a radius which is approx. equal to that of the outer shell of the cylindrical body. An X-ray-opaque marker 45a or 45b is disposed at the level of the shoulder 44a or 44b respectively in order to localize the balloon 4, especially in order to ensure that the balloon is disposed in the region of the thrombus prior to inflation.

The balloon body 40 has openings 43 distributed along the entire length l or at least over an effective region le which is at least 75% of the length l.

As shown in the detailed view in FIG. 7, these have a diameter d of between 10 μm and 100 μm. When the internal pressure is increased, the diameter d increases in such a way that at a certain threshold pressure the delivered medication or the solution can slowly emerge atraumatically.

The number of openings averages at least 25 openings per cm², preferably more than 50 openings per cm², preferably more than 200 openings per cm², preferably more than 500 openings per cm², preferably more than 1000 openings per cm², relative to the entire surface area of the dilated balloon body 40.

Preferably, the arrangement of the openings can also be determined by their average spacing ai, aj, . . . . The spacing ai, aj, . . . between the openings 43 averages less than 2 mm, preferably less than 1.4 mm, especially less than 0.7 mm, especially preferably less than 0.45 mm.

The total number of openings formed in the balloon body 40 is over 50 openings and can even be 100 openings or more. These can be distributed more or less homogeneously over the wall 41.

Due to the small diameter of the openings 43, the internal pressure can—despite the high number of openings—be substantially increased, without too much medication being administered in a short time and without opposing the build-up of a high internal pressure. By virtue of the increased internal pressure, the balloon can also effect greater widening of the vessels and stronger pressing against the vessel wall.

A suitable material for the production of the wall and/or skin 41 of the balloon body 40/the balloon 4 is, e.g. nylon.

In a non-dilated state, the diameter h is so small that the balloon can be disposed in the first lumen 21 of the aspiration catheter 2 and can be moved and/or passed through the first lumen 20 of the aspiration catheter 2. The size of the aspiration catheter and/or the cross-sectional area of the first lumen 20 must therefore be adapted to the cross-sectional area of the non-dilated balloon 4, i.e. the balloon body cross-section must especially be smaller than or lie within the order of magnitude of the cross-section of the first lumen 21.

The medication is intended to be administered as atraumatically as possible. In order that the internal pressure may be substantially increased, a pressure syringe is used as a rule instead of a normal (manual) syringe. A pressure syringe allows not only larger pressures but also defined pressures to be generated. As a result, the balloon dilates much more and a greater pressure, e.g. up to 10 bar, is exerted on the arterial wall. As a result, the liquid is administered very selectively and plaques and thrombi on the vessel wall are better dissolved. The balloon pressing against the wall firmly holds loose plaque parts so that they cannot be transported into other regions of the body.

By virtue of this type of administration, multiple receptor binding is achieved as compared with intravenous administration. Administration can also be implemented locally in a targeted manner. Administration takes place, e.g., for one minute. The arterial volume in the heart region is 1-2 ml. It is rinsed with 10 ml saline solution or medication.

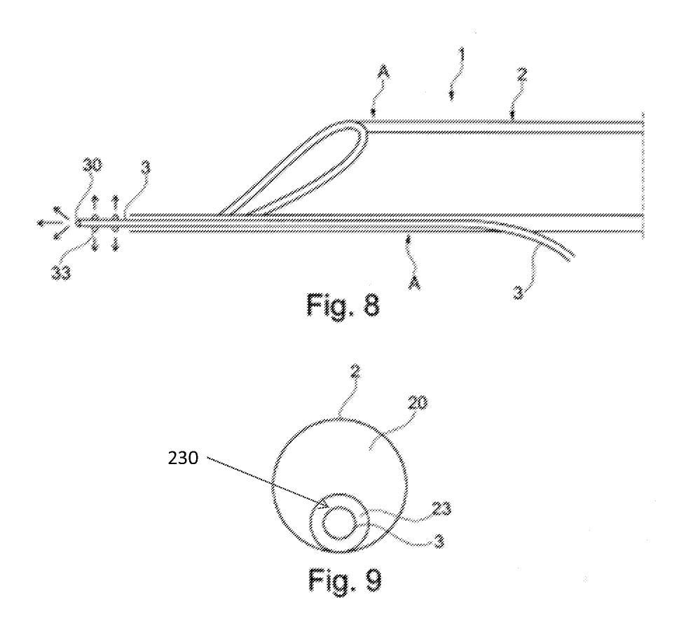

An alternative embodiment of the inventive system 1 is shown in FIG. 8.

In contrast to the first embodiment, system 1 does not have a balloon 4 in this case. Instead, a liquid is administered via the guide member 3 of the aspiration catheter 2.

As shown in FIG. 9, the aspiration catheter 2 has a first lumen 20 (aspiration lumen) and a second lumen 23 for guiding the guide member 3.

In the guide member 3, there extends between the proximal end (not shown) and the distal end region 30 which protrudes beyond the opening 230 of the second lumen 23, a third lumen, through which the medication M is administered in the second phase of administration. For this purpose, the distal end region 30 of the guide member 3 has, e.g., openings 33, through which the liquid can enter the blood vessel B. In this case, the guide member 3 is configured as a microcatheter or infusion catheter with the third lumen for the purpose of delivering the medication to the openings 33 disposed in the distal region 30.

A cross-section A-A from FIG. 8 is shown in FIG. 9 for clarity. In this embodiment, the first lumen 20 serves exclusively to aspirate the dissolved thrombotic material D (aspiration lumen 20). The second lumen 23 serves to fixedly accommodate the guide member 3. The latter has a third lumen for the administration of a liquid M.

Application and the description of the first embodiment are also transferable to the second embodiment.

1. A system for removing thrombotic material from a blood vessel comprising an aspiration catheter and an infusion member, wherein the infusion member has a delivery catheter configured for delivering a fluid and an expandable balloon connected to the delivery catheter, wherein the balloon has at least one of openings and a permeable wall region or a semi-permeable wall region through which the fluid can pass, wherein
the infusion member can be movably accommodated in and moved through a first lumen of the aspiration catheter.

2. The system in accordance with claim 1, wherein the openings and the permeable wall region or the semi-permeable wall region, respectively, are configured such that atraumatic administration of the fluid takes place after a predetermined fluid pressure has been reached.

3. The system in accordance with claim 2, wherein the infusion member is disposed so as to be axially displaceable in the first lumen of the aspiration catheter.

4. The system in accordance with claim 3, wherein the openings have a diameter between 20 μm and 50 μm.

5. The system in accordance with claim 4, wherein the balloon has a plurality of openings with an average density per unit area of at least 1000 openings per cm².

6. The system in accordance with claim 5, wherein the balloon has at least 1000 openings.

7. The system in accordance with claim 1, wherein the aspiration catheter has a proximal end and a distal end as well as a longitudinal axis, and the distal end is beveled.

8. The system in accordance with claim 1, wherein the system has a guide member which is fixed to the aspiration catheter.

9. The system in accordance with claim 1, wherein the aspiration catheter has a second lumen in which the guide member is embedded at least sectionwise.

10. A balloon for a system in accordance with claim 1, wherein the balloon has a plurality of openings which have a diameter between 20 μm and 50 μm.

11. The balloon in accordance with claim 10, wherein the balloon has a plurality of openings with an average surface density of at least 1000 openings per cm².

12. The balloon in accordance with claim 10, wherein the balloon has at least 1000 openings

13. (canceled)

14. The balloon for a system in accordance with claim 10, wherein the balloon has a plurality of openings which have a diameter between 10 μm and 100 μm.

15. The balloon in accordance with claim 10, wherein the balloon has a plurality of openings with an average surface density of at least 25 openings per cm².

16. The balloon in accordance with claim 10, wherein the balloon has at least 50 openings.

17. The system in accordance with claim 1, wherein the infusion member is disposed so as to be axially displaceable in the first lumen of the aspiration catheter.

18. The system in accordance with claim 1, wherein the openings have a diameter between 10 μm and 100 μm.

19. The system in accordance with claim 1, wherein the balloon has a plurality of openings with an average density per unit area of at least 25 openings per cm².

20. The system in accordance with claim 1, wherein the balloon has at least 50 openings.

21. A system for the removal of thrombotic material from a blood vessel comprising an aspiration catheter which has a first lumen for aspirating thrombotic material from a blood vessel and a guide member for introducing the aspiration catheter into the blood vessel, wherein the guide member has a proximal end and a distal end, as well as a third lumen through which liquids can be transported from the proximal end of the guide member to the distal end of the guide member.
